(19) United States
(12) Patent Application Publication
(10) Pub. No.: US 2022/0265619 A1
(43) Pub. Date: Aug. 25, 2022

(54) COMBINATION TREATMENT OF LIVER DISEASES USING FXR AGONISTS

(71) Applicant: Novartis AG, Basel (CH)

(72) Inventors: Dominique BREES, Arlesheim (CH); Patricia LOPEZ, Basel (CH)

(21) Appl. No.: 17/628,848

(22) PCT Filed: Jul. 21, 2020
(86) PCT No.: PCT/IB2020/056837

§ 371 Date: Jan. 20, 2022

Related U.S. Application Data
(60) Provisional application No. 62/877,452, filed on Jul. 23, 2019.
(60) Provisional application No. 62/901,427, filed on Sep. 17, 2019.
(60) Provisional application No. 62/980,773, filed on Feb. 24, 2020.

(51) Int. Cl. A61K 31/439; A61K 31/575; A61K 31/4162; A61K 31/4439; A61K 31/454; A61K 31/496; A61K 31/7042; A61K 31/351; A61K 31/7048; A61K 31/7056; A61P 1/16

(52) U.S. Cl. CPC A61K 31/439; A61K 31/575; A61K 31/4162; A61K 31/4439; A61K 31/454; A61K 31/496; A61K 31/7042; A61K 31/351; A61K 31/7048; A61K 31/7056; A61P 1/16

(57) ABSTRACT

The present invention relates to combinations for treating, preventing, or ameliorating conditions mediated by farnesoid X receptors (FXRs), in particular liver diseases or intestinal disease, comprising administering to a subject in need thereof a therapeutically effective amount of an FXR agonist.

### FIELD OF THE INVENTION

The present invention relates to combinations for treating, preventing, or ameliorating conditions mediated by farnesoid X receptors (FXRs), in particular liver diseases or intestinal disease, comprising administering to a subject in need thereof a therapeutically effective amount of an FXR agonist.

### BACKGROUND OF THE INVENTION

Nonalcoholic fatty liver disease (NAFLD) is the most common cause of chronic liver disease in the Western world. The main stages of NAFLD are: 1-simple fatty liver (steatosis); 2-non-alcoholic steatohepatitis (NASH), a more serious form of NAFLD with fat accumulation accompanied by inflammation and cell injury; 3-fibrosis, where there is a persistent inflammation in the liver resulting in the generation of fibrous scar tissue around the liver cells and blood vessels; and 4-cirrhosis, where damage is permanent and can lead to liver failure and liver cancer (hepatocellular carcinoma).

Liver transplantation is the only treatment for advanced cirrhosis with liver failure. Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2024. NASH is highly associated with the metabolic syndrome and Type 2 diabetes mellitus. Furthermore, cardiovascular mortality is an important cause of death in NASH patients.

Obesity has become a major global health problem that contributes causally to and exacerbates many serious co-morbidities including hypertension, dyslipidemia, type 2 diabetes (T2DM) and importantly non-alcoholic fatty liver disease (NAFLD). In support of the link between obesity and fatty liver linked hepatic injury, weight loss either through bariatric surgery, diet or exercise leads to improvement in histologic NASH. This suggests that targeting obesity in NASH patients is likely to limit or reverse liver disease progression. A novel mechanism to lower body weight is via inhibition of the sodium glucose co-transporters 1 and 2 (SGLTs) resulting in inhibition of the glucose absorption in the gut and reabsorption in the kidney.

Development of NASH, involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

When tested in NASH patients, obeticholic acid (OCA), a bile acid mimetic, showed efficacy, in particular a significant improvement in NAS, i.e. strong impact on steatosis with additional effects on lobular inflammation and ballooning. But OCA long term administration raises safety concerns because it was associated with pruritus, as well as with lipid abnormalities, i.e. increased low density lipoprotein (LDL) cholesterol (see Results from REGENERATE (NCT02548351), A Phase 3 International, Randomized, Placebo-Controlled Study Evaluating Obeticholic Acid Treatment for NASH, EASL 2019 Apr. 10-14 Vienna). Pruritus is the most common adverse effect in the patients treated with OCA. This side effect reported in association with the treatment with the FXR agonist OCA may be requiring dose adjustment and/or discontinuation of the administration. Pruritus may also be managed in most patients by i.e. use of bile acid sequestrants, antihistamines, dose reduction, or symptomatic treatment. Furthermore, to avoid the risk of adverse cardiovascular events, concomitant administration of statins may be required for long-term treatment of NASH patients treated with OCA.

Currently there is no approved therapy for NASH. Therefore, there is a need to provide treatments for fibrotic/cirrhotic diseases or disorders, e.g. liver diseases or disorders, e.g. NASH, which can address the different aspects of these complex conditions, while demonstrating an acceptable safety and/or tolerability profile.

### SUMMARY OF THE INVENTION

The invention relates to pharmaceutical combinations for treating, preventing, or ameliorating conditions mediated by FXRs, in particular liver diseases or intestinal diseases, e.g. NASH, comprising an FXR agonist and an SGLT inhibitor. Furthermore, the present invention is directed to a pharmaceutical combination comprising the farnesoid X receptors (FXRs) agonist tropifexor and licogliflozin, optionally in the presence of a pharmaceutically acceptable carrier, and pharmaceutical compositions comprising them.

Tropifexor is a highly potent FXR agonists that is currently tested in nonalcoholic steatohepatitis patients with fibrosis (NCT02855164 study; Tully et al, J Med Chem 2017;60:9960-9973). Tropifexor (also known as LJN452), was described in WO 2012/087519 (Example 1, compound 1-IB), and has the chemical structure

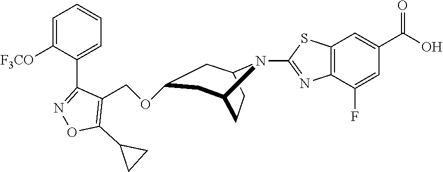

i.e., 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid).

Licoglifozin is a potent inhibitor of the sodium glucose co-transporters (SGLTs) 1 and 2 that decreases absorption of glucose in the gut and reabsorption in the kidney. Licogliflozin (also known as LIK066), was described in WO 2011/048112 (Example 62), has the chemical structure

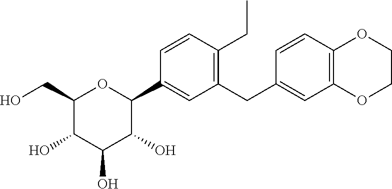 [SMILES: CCc1ccc([C@@H]2O[C@H](CO)[C@@H](O)[C@H](O)[C@H]2O)cc1Cc1ccc2c(c1)OCCO2]

i.e., (2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol. Licogliflozin was found to be safe and tolerated, had a favorable pharmacokinetic profile, and resulted in up to 3% placebo-adjusted weight loss over just 2 weeks in both healthy subjects and patients with T2DM. Licogliflozin at 150 mg daily dose results in a significant weight loss in obese patients (˜6%) after 12 week treatment. Furthermore, twelve week treatment with licogliflozin at 150 mg once daily, in normoglycemic and dysglycemic subjects was generally safe and well tolerated with diarrhea observed as a dose-limiting toxicity.

A combination of tropifexor and licogliflozin has the potential to address metabolic, anti-inflammatory and antifibrotic pathways involved in NASH. Tropifexor and licogliflozin impact distinct targets affecting different nodes of NASH pathophysiology as evidenced by:
i. Tropifexor activates a nuclear receptor (FXR) that has pleiotropic downstream effects in the liver.
ii. Licogliflozin inhibits two closely related glucose cotransporters (SGLT1/2) in the gut and kidney.
iii. Both compounds are potent and highly specific for their targets
iv. FXR is not associated with changes in SGLT1 or SGLT2 expression or activity, and there is no known downstream intersection of the two pathways
v. Tropifexor specifically increases the enterocyte hormone Fibroblast growth factor 19 (FGF19) that has beneficial metabolic and anti-inflammatory effects. No such effect on FGF19 has been described for licogliflozin.

In one aspect, the invention provides a pharmaceutical combination comprising, separate or together, an FXR agonist and an SGLT inhibitor, for simultaneous, sequential, or separate administration.

In another aspect, the invention provides new treatment regimens for pharmaceutical combinations containing (i) at least one FXR agonist, such as for example tropifexor, wherein the administration of the FXR agonist is occurring in the evening., and (ii) an SGLT 1 and/or 2 inhibitor. The treatment regimens according to the present invention offer the benefit of a high therapeutic efficacy while having low incidence of side effects, such as itching and/or lipid abnormalities (e.g. increased LDL cholesterol), which are, observed while using conventional treatment regimen. These treatment regimens further provide subjects with a convenient once daily dosing, thus supporting patient compliance.

### BRIEF DESCRIPTION OF DRAWINGS

FIG. 1 provides the study design of a 2 week study in Cynomolgus monkey treated with the FXR agonist LJP305 (compound described in Tully et al, J Med Chem 2017;60:9960-9973).

FIG. 2 shows the 7α-hydroxy-4-cholesten-3-one (C4) measurements in the different groups of the 2 week study in Cynomolgus monkey treated with the FXR agonist LJP305.

FIG. 3 shows the cholic acid (CA) measurements in the different groups of the 2 week study in Cynomolgus monkey treated with the FXR agonist LJP305.

FIG. 4 shows the levels of Chenodeoxycholic acid (CDCA) in the different groups of the 2 week study in Cynomolgus monkey treated with the FXR agonist LJP305.

FIG. 5 shows that in vitro human hepatocytes treated with the FXR agonists OCA and cilofexor have decreased LDL uptake.

### DETAILED DESCRIPTION OF THE INVENTION

In one aspect, the invention provides new pharmaceutical combinations, containing, separate or together, (i) the FXR agonist 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid, in free form or a pharmaceutically acceptable salt, solvate, prodrug, ester and/or an amino acid conjugate thereof, also known under its INN tropifexor; and (ii) licogliflozin (as herein defined, e.g. in free form or as a pharmaceutically acceptable salt thereof) for simultaneous, sequentially or separate administration. The pharmaceutical combinations comprise: (i) an amount of 120 μg to about 250 μg, of about 140 μg to about 200 μg of tropifexor, and (ii) an amount of about 30 mg or of about 150 mg of licogliflozin. The pharmaceutical combinations comprise: (i) an amount of about 140 μg of tropifexor, and (ii) an amount of about 30 mg of licogliflozin. There is also provided a medicament, comprising such combinations.

In another aspect, the invention provides a method for the treatment of a condition mediated by Farnesoid X receptor (FXR), in particular a liver disease or an intestinal disease, in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

It has been found that administering an FXR agonist to a subject in need thereof in the evening, for example shortly before or at bedtime, is beneficial for therapeutic efficacy and for safety (such as reducing itch and/or lipid abnormalities).

7α-hydroxy-4-cholesten-3-one (C4) is an intermediate bile acid precursor directly produced by cholesterol 7-alpha-monooxygenase or cytochrome P450 7A1 (Cyp7A1). C4 has 2 peaks in the plasma, one around 1 pm and the other around 9 pm (Galman et al, Gastroenterology 2005; 129:1445-1453). These peaks are corresponding to timing of the larger meals of the day; the bile acids being needed for digestion. This implies that Cyp7A1, which produces C4, as well as FXR, which is the counter mechanism for the production, are following the same daily rhythms in human. Administration of an FXR agonist in the evening (at/after 9 pm), should allow the FXR agonist to stimulate the system when the activity of the transcription factor FXR is decreasing hence allowing for a more prolonged effect of FXR agonist during the night when normally FXR activity is at the lowest. Such a dosing schedule should increase the efficacy of the FXR agonist.

Chenodeoxycholic acid (CDCA), major primary bile acid, is primarily responsible for the bile acid induced itch (Alemi et al, The Journal of Clinical Investigation 2013; 123:1513-1530). It has been found that the FXR agonist-induced itch is caused by a sustained inhibition of Cyp7a1 causing a shutdown of the C4/bile acid production leading to the activation of the alternate bile acid pathway via activation of Cyp27a1, this leading to the production of prurigenic CDCA bile acid. Administration of an FXR agonist when the enzymatic activity of Cyp7A1 is at the lowest should minimize the effect of FXR-mediated inhibition of Cyp7A1 and consequent activation of the alternate bile acid pathway.

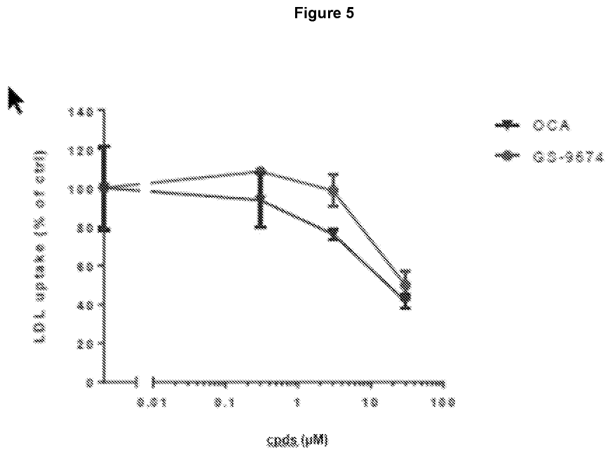

In addition, FXR agonist treatments have been associated with lipid abnormalities, including increases in peripheral LDL (Neuschwander-Tetri et al, The Lancet 2015; 385: 956-965). The reduction of the bile acid pathway by FXR agonists could lead to intracytoplasmic increase in cholesterol in the hepatocytes. Increase cholesterol in hepatocytes is associated with a counter mechanism of decrease LDL receptor on the surface of the cells (Goldstein et al Circulation. 1987 Sep;76(3):504-7). Such a decrease in the LDL receptor on the surface of the hepatocytes will ultimately result in an increase in circulating LDL; the phenotype observed in the clinics. We have demonstrated in vitro, using in vitro human hepatocytes, that FXR agonists reduce the LDL uptake by hepatocytes in a dose dependent manner (FIG. 5). Those data indicates that blocking the Cyp7A1 and the bile acid pathway leads to the peripheral increase in LDL. To mitigate the increase in circulating LDL, it is proposed to administer an FXR agonist to the subjects in need thereof in the evening (e.g. from about 6 pm to about 12 pm, preferably from about 8 pm to about 11 pm, preferably around 9 pm) to reduce the impact of the FXR agonist on circulating LDL.

Various (enumerated) embodiments of the present invention are described herein. It will be recognized that features specified in each embodiment may be combined with other specified features to provide further embodiments of the present disclosure.

### Embodiments (a)

1 a. A pharmaceutical combination for simultaneous, sequentially or separate administration, comprising (i) an FXR agonist, wherein the FXR agonist is administered once daily at a therapeutically effective dose, and wherein the FXR agonist is administered in the evening; and (ii) an SGLT inhibitor, e.g. SGLT 1/2 inhibitor.

2a: A pharmaceutical combination for simultaneous, sequential or separate administration, comprising (i) an FXR agonist selected from tropifexor, obeticholic acid, nidufexor, cilofexor, TERN-101, EDP-305, PXL007, AGN242266 and MET409; and (ii) an SGLT inhibitor, e.g. SGLT 1/2 inhibitor.

3a. The pharmaceutical combination according to Embodiment 1a or 2a, wherein the FXR agonist is tropifexor, in free form or a pharmaceutically acceptable salt, solvate, prodrug, ester and/or an amino acid conjugate thereof.

4a. The pharmaceutical combination according to Embodiment 3a, comprising about 90 μg to about 250 μg of tropifexor.

5a. The pharmaceutical combination according to Embodiment 3a, comprising about 140 μg to about 200 μg of tropifexor.

6a. The pharmaceutical combination according to Embodiment 3a, comprising about 120 μg to about 250 μg of tropifexor.

7a. The pharmaceutical combination according to Embodiment 3a, comprising about 140 μg of tropifexor.

8a. The pharmaceutical combination according to any one of Embodiments 1a to 7a, wherein the SGLT inhibitor is selected from licogliflozin, dapagliflozin, canagliflozin, empagliflozin, ipragliflozin, ertugliflozin, mizagliflozin, sotagliflozin.

9a. The pharmaceutical combination according to Embodiment 8a, wherein the SGLT inhibitor is licogliflozin, in free form, or as a pharmaceutically acceptable salt or crystalline form thereof.

10a. The pharmaceutical combination according to Embodiment 9a, comprising about 20 to about 150 mg of licogliflozin.

11a. The pharmaceutical combination according to Embodiment 9a, comprising about 30 mg or about 150 mg of licogliflozin. 12a. The pharmaceutical combination according to Embodiment 9a, comprising about 20 mg, about 30 mg, about 40 mg, about 50 mg, about 60 mg, about 70 mg, about 80 mg, about 90 mg, about 100 mg, or about 120 mg of licogliflozin.

13a. The pharmaceutical combination according to Embodiment 9a, comprising about 30 mg to about 50 mg of licogliflozin.

14a. The pharmaceutical combination according to Embodiment 9a, comprising about 30 mg of licogliflozin.

15a. A pharmaceutical combination for simultaneous, sequential or separate administration, comprising: (i) about 90 μg of tropifexor; and (ii) about 30 mg of licogliflozin.

16a. A pharmaceutical combination for simultaneous, sequential or separate administration, comprising: (i) about 140 μg of tropifexor; and (ii) about 30 mg of licogliflozin.

17a. The pharmaceutical combination according to any one of Embodiments 1a to 16a, comprising tropifexor in free form.

18a. The pharmaceutical combination according to any one of Embodiments 1a to 17a, comprising an L-proline salt of licoglifozin.

19a. The pharmaceutical combination according to any one of Embodiments 1a to 17a, comprising a crystalline form of licogliflozin.

20a. The pharmaceutical combination according to Embodiment 19a, wherein said licogliflozin is an L-proline co-crystal of licogliflozin.

21a. The pharmaceutical combination according to Embodiment 20a, wherein the L-proline co-crystal of licogliflozin has a 1:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol.

22a. The pharmaceutical combination according to Embodiment 20a, wherein the L-proline co-crystal of licogliflozin has a 2:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol (also known as (S)-pyrrolidine-2-carboxylic acid compound with (2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol (2:1)).

23a. The pharmaceutical combination according to any one of Embodiments 1a to 22a, wherein said combination is a fixed combination.

24a. The pharmaceutical combination according to any one of Embodiments 1a to 22a, wherein said combination is a free combination.

25a. A pharmaceutical combination according to any one of Embodiments 1a to 24a, for use in preventing, delaying or treating a condition mediated by Farnesoid X receptor, in particular a liver disease or an intestinal disease.

26a. A method of preventing, delaying or treating a liver disease or disorder, in a subject in need thereof, comprising administering a therapeutically effective amount of the pharmaceutical combination according to any one of Embodiments 1a to 24a.

27a. The method according to Embodiment 26a, wherein the liver disease or disorder is a fibrotic or cirrhotic liver disease or disorder, selected from the group consisting of non-alcoholic fatty liver disease (NAFLD), non-alcoholic steatohepatitis (NASH), liver cirrhosis, alcohol-induced cirrhosis, cystic fibrosis-associated liver disease (CFLD), liver fibrosis, and progressive fibrosis of the liver caused by any of the diseases above or by infectious hepatitis.

28a. The method according to Embodiment 27a, wherein the liver disease or disorder is non-alcoholic fatty liver disease (NAFLD), non-alcoholic steatohepatitis (NASH), primary biliary cirrhosis (PBC), liver fibrosis, or liver cirrhosis.

29a. The method according to Embodiment 27a, wherein the liver disease or disorder is non-alcoholic fatty liver disease, (NAFLD).

30a. The method according to Embodiment 27a, wherein the liver disease or disorder is non-alcoholic steatohepatitis (NASH).

31a. The method according to Embodiment 30a, further comprising resolution of steatohepatitis.

32a. The method according to Embodiment 27a, wherein the liver disease or disorder is liver fibrosis.

33a. The method according to any one of Embodiments 31a to 32a, further comprising improvement in liver fibrosis.

34a. The method according to any one of Embodiments 26a-33a, wherein the FXR agonist is administered in the evening.

35a. The method according to Embodiment 34a, thereby reducing the risk pruritus, associated with the administration of the FXR agonist.

36a. The method according to Embodiment 35a, thereby reducing the risk of lipid abnormality, associated with the administration of the FXR agonist.

37a. The method according to any one of Embodiments 26a-33a, wherein the SGLT inhibitor is administered in the evening.

38a. The method according Embodiment 37a, thereby reducing the risk of diarrhea associated with the administration of the SGLT inhibitor.

### Embodiments (b)

1 b. A method for the treatment of a condition mediated by Farnesoid X receptor (FXR), in particular a liver disease or an intestinal disease, in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

2b. A method for the prevention of a condition mediated by Farnesoid X receptor (FXR), in particular a liver disease or an intestinal disease, in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

3b. A method for the treatment, stabilization or lessening the severity or progression of a non-alcoholic fatty liver disease (NAFLD) in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

4b. A method for the treatment, stabilization or lessening the severity or progression of an intestinal disease in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

5b. A method for the treatment, stabilization or lessening the severity or progression of a non-alcoholic steatohepatitis (NASH) in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

6b. A method for slowing, arresting, or reducing the development of a chronic liver disease or disorder, e.g. NAFLD, NASH, liver fibrosis or PBC, in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

7b. A method for reducing cirrhosis or fibrosis in a subject having a disease that is non-alcoholic fatty liver disease (NAFLD) or non-alcoholic steatohepatitis (NASH), comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor.

8b. The method according to any one of Embodiments 1b to 7b, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's Steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to Cirrhosis, inhibiting progression of Non-Alcoholic Fatty Liver Disease (NAFLD) and/or Non-Alcoholic Steatohepatitis (NASH), or any combination thereof.

9b. The method according to any one of Embodiments 1b to 8b, wherein the FXR agonist is selected from tropifexor, obeticholic acid, nidufexor, cilofexor, TERN-101, EDP-305, PXL007, AGN242266 and MET409.

10b. The method according to Embodiment 9b, wherein the FXR agonist is obeticholic acid.

11b. The method according to Embodiment 10b, wherein obeticholic acid is administered at a daily dose of about 5 mg, of about 10 mg, of about 15 mg, of about 20 mg, of about 25 mg, of about 30 mg, of about 40 mg or of about 50 mg.

12b. The method according to Embodiment 9b, wherein the FXR agonist is tropifexor.

13b. The method according to Embodiment 12b,wherein tropifexor is administered at a daily dose of about 90 μg to about 250 μg, e.g. of about 140 μg to about 200 μg.

14b. The method according to Embodiment 12b, wherein tropifexor is to be administered at a dose of about 90 μg/day, of about 140 μg/day, of about 150 μg/day, of about 160 μg/day, of about 170 μg/day, of about 180 μg/day, of about 190 μg/day, of about 200 μg/day, of about 210 μg/day, of about 220 μg/day, of about 230 μg/day, of about 240 μg/day or of about 250 μg/day.

15b. The method according to Embodiment 12b wherein tropifexor is to be administered at a daily dose of about 140 μg.

16b. The method according to any one of Embodiments 1 b to 15b, wherein the SGLT inhibitor is selected from licogliflozin, dapagliflozin, canagliflozin, empagliflozin, ipragliflozin, ertugliflozin, mizagliflozin, sotagliflozin.

17b. The method according to Embodiment 16b, wherein the SGLT inhibitor is licogliflozin.

18b. The method according to Embodiment 17b, wherein said licoglifozin is an L-proline salt of licoglifozin.

19b. The method according to Embodiment 17b, wherein said licoglifozin is a crystalline form of licogliflozin.

20b. The method according to Embodiment 19b, wherein said licogliflozin is an L-proline co-crystal of licogliflozin.

21b. The method according to Embodiment 20b, wherein the L-proline co-crystal of licogliflozin has a 1:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol.

22b. The method according to Embodiment 20b, wherein the L-proline co-crystal of licogliflozin has a 2:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol (also known as (S)-pyrrolidine-2-carboxylic acid compound with (2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol (2:1)).

23b. The method according to any one of Embodiments 17b to 22b, wherein ii) licogliflozin is administered at an amount of about 30 mg or of about 150 mg.

24b. The method according to any one of Embodiments 1b to 23b, wherein said evening administration reduces the risk of pruritus associated with administration of the FXR agonist.

25b. The method according to any one of Embodiments 1b to 23b, wherein said evening administration reduces the risk of lipid abnormality, associated with administration of the FXR agonist.

26b. The method according to any one of Embodiments 1b to 23b, wherein said administration comprises resolution of steatohepatitis, e.g. NASH.

27b. The method according to any one of Embodiments 1b to 23b, wherein said administration comprises improvement in liver fibrosis.

28b. The method according to any one of Embodiments 1b to 23b, wherein said administration comprises resolution of steatohepatitis, e.g. NASH, improvement in liver fibrosis, or a combination thereof.

29b. The method according to any one of Embodiments 1 b to 28b wherein (ii) the SGLT inhibitor is administered once daily, e.g. in the evening.

30b. The method according to any one of Embodiment 29b, wherein said administration reduces the risk of diarrhea, associated with the administration of the SGLT inhibitor.

In another aspect, the invention provides a medicament, comprising such combinations.

In yet another aspect, the invention provides the use of tropifexor, in combination, e.g. fixed or free combination, with licogliflozin (or a pharmaceutically acceptable salt, solvate, prodrug and/or ester thereof), for the manufacture of a medicament for the prevention or treatment of a liver disease or disorder, e.g. a liver disease or disorder selected from the group consisting of NAFLD, NASH, hepatosteatosis, liver fibrosis, cirrhosis, PBC. Components (i) and (ii) can be administered together, one after the other or separately in one combined unit dose form or in two separate unit dose forms.

In yet another aspect, the invention provides a pharmaceutical combination according to any one of above listed Embodiments, for treating or preventing non-alcoholic steatohepatitis (NASH), and wherein NASH is mild to moderate with fibrosis level F2-F3.

In yet another aspect, the invention provides the use of tropifexor, in combination, e.g. fixed or free combination, with licogliflozin (or a pharmaceutically acceptable salt, solvate, prodrug and/or ester thereof), for the manufacture of a medicament for the prevention or treatment of a liver disease or disorder, e.g. a liver disease or disorder selected from the group consisting of NAFLD, NASH, hepatosteatosis, liver fibrosis, cirrhosis, PBC.

In yet another aspect, the invention provides an FXR agonist in combination, e.g. fixed or free combination, with an SGLT inhibitor, e.g. SGLT 1/2 inhibitor, a method, a pharmaceutical composition, or a use, according to any one of above listed Embodiments, wherein NASH is confirmed based on liver biopsy (also called biopsy-proven NASH) and NASH is mild to moderate with fibrosis level F2-F3.

In yet another aspect, the invention provides an FXR agonist in combination, e.g. fixed or free combination, with an SGLT inhibitor, e.g. SGLT 1/2 inhibitor, according to any one of the above listed Embodiments, wherein presence of NASH has been demonstrated by:

i) Histologic evidence of NASH based on liver biopsy obtained 2 years or less before treatment with an FXR agonist according to any one of the above Embodiments, with a diagnosis consistent with NASH, fibrosis level F1, F2, F3 or F4, no diagnosis of alternative chronic liver diseases, or

ii) Phenotypic diagnosis of NASH, or

iii) Noninvasive, disease-specific biomarkers.

In some aspects, the pharmaceutical combinations as defined herein, are provided for the treatment of a disease or disorder mediated by FXR, e.g. a liver disease or disorder, e.g. a chronic liver disease or disorder, e.g. a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatosis or PBC.

In yet another aspect, a pharmaceutical unit dosage form composition comprising about 90 μg, about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration once daily, in the evening, or shortly before or at bedtime. Such unit dosage form compositions may be in a form selected from a liquid, a tablet, a capsule. Also these unit dosage form compositions are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH), e.g. for use in treating phenotypic non-alcoholic steatohepatitis (NASH).

In yet another aspect, the pharmaceutical combinations as defined herein are provided for preventing or delaying progression of a chronic liver disease or disorder to a more advanced stage or a more serious condition thereof, e.g. for preventing or delaying progression of a chronic liver disease or disorder selected from the group consisting of NAFLD, NASH, hepatic fibrosis and PBC.

### Definitions

For purposes of interpreting this specification, the following definitions will apply and whenever appropriate, terms used in the singular will also include the plural and vice versa.

As used herein, the term “about” in relation to a numerical value x means+/−10%, unless the context dictates otherwise.

As used herein, a “SGLT inhibitor” refer to any to any agent that is capable of inhibiting SGLT, e.g. individual SGLT1 and SGLT2 inhibitors, as well as dual SGLT1/2 inhibitors. The SGLT inhibitor as used herein refers, for example, to licogliflozin, dapagliflozin, canagliflozin, empagliflozin, ipragliflozin, ertugliflozin, mizagliflozin, sotagliflozin.

Sotagliflozin is (2S,3R,4R,5S,6R)-2-[4-chloro-3-[(4-ethoxyphenyl)methyl]phenyl]-6-methylsulfanyloxane-3,4,5-triol, is also known as LX4211.

As used herein, a “FXR agonist”/“FXR agonists” refer to any agent that is capable of binding and activating farnesoid X receptor (FXR) which may be referred to as bile acid receptor (BAR) or NR1 H4 (nuclear receptor subfamily 1, group H, member 4) receptor. FXR agonist may act as agonists or partial agonists of FXR. The agent may be e.g. a small molecule, an antibody or a protein, preferably a small molecule. The activity of an FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, WO2017/143134.

The FXR agonist is preferably selected from: tropifexor, nidufexor, obeticholic acid (6α-ethyl-chenodeoxycholic acid), cilofexor (GS-9674, Px-102),

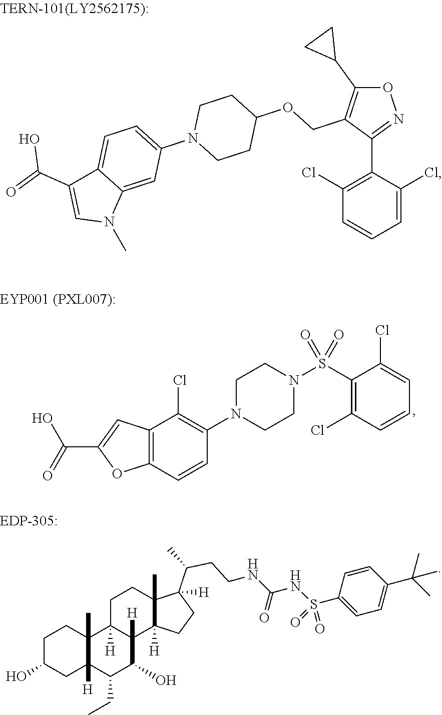

As used herein, the terms “salt” or “salts” refer to an acid addition or base addition salt of a compound of the invention. “Salts” include in particular “pharmaceutical acceptable salts”, and both can be used interchangeably herein.

As used herein, the term “amino acid conjugate” when used in relation to tropifexor refers to conjugates of tropifexor with any suitable amino acid. Preferably, such suitable amino acid will have the added advantage of enhanced integrity in bile or intestinal fluids. Suitable amino acids include but are not limited to glycine, taurine and acylglucuronide conjugates of tropifexor.

As used herein, the term “pharmaceutically acceptable” means a nontoxic material that does not substantially interfere with the effectiveness of the biological activity of the active ingredient(s).

As used herein, the term “prodrug” refers to a compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the term “co-crystal” refers to crystalline materials composed of two or more different molecules, typically active pharmaceutical ingredient (API) and co-crystal formers, in the same crystal lattice. (US Food and Drug Administration, Regulatory Classification of Pharmaceutical Co-Crystals Guidance for Industry, February 2018 Revision 1).

As used herein, the terms “subject” or “subjects” refer to a mammalian organism, preferably a human being, who is diseased with the condition (i.e. disease or disorder) of interest and who would benefit from the treatment, e.g. a patient.

As used herein, a subject is “in need of” a treatment if such subject would benefit biologically, medically or in quality of life from such treatment.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “nonalcoholic fatty liver disease” (NAFLD) may refer to non-alcoholic fatty liver (NAFL), non-cirrhotic NASH, and NASH with cirrhosis.

For example, “treating” NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation, e.g. slow the progress of, halt, or reverse disease progression and improve clinical outcomes (i.e., prevent progression to cirrhosis and 283 cirrhosis complications, reduce the need for liver transplantation, and improve survival)

Also “treating” NASH may refer to slow the progress of, halt, or reverse disease progression and improve clinical outcomes i.e., prevent progression to cirrhosis and Resolution of steatohepatitis and no worsening of liver fibrosis on NASH clinical research network (CRN) histological score.

The treatment of NASH includes:

“Resolution of steatohepatitis” is defined as absence of fatty liver disease or isolated or simple steatosis without steatohepatitis and a NAS score of 0-1 for inflammation, 0 for ballooning, and any value for steatosis; cirrhosis complications, reduction in the need for liver transplantation, and improved survival; or

Improvement in liver fibrosis greater than or equal to one stage (NASH CRN histological score) and no worsening of steatohepatitis (e.g. defined as no increase in NAS for ballooning, inflammation, or steatosis); or

Both resolution of steatohepatitis and improvement in fibrosis (as defined above). “Treating” or “treatment” of NAFLD or NASH in a human includes one or more of:
a) Reducing the risk of developing NAFLD or NASH, i.e., causing clinical symptoms of NAFLD or NASH not to develop in a subject who may be predisposed to NAFLD or NASH
b) Inhibiting NAFLD or NASH, i.e., arresting or reducing the development of NALFD or NASH or its clinical symptoms; and
c) Relieving NAFLD or NASH, i.e., causing regression, reversal, or amelioration of the NAFLD or NASH or reducing number, frequency, duration or severity of its clinical symptoms.

As used herein, the terms “prevent”, “preventing” or “prevention” of any disease or disorder refers to the prophylactic treatment of the disease or disorder; or delaying the onset or progression of the disease or disorder

As used herein, the term “therapeutically effective amount” refers to an amount of the compound, which is sufficient to achieve the stated effect. Accordingly, a therapeutically effective amount used for the treatment or prevention of a liver disease or disorder, as hereinabove defined, is an amount sufficient for the treatment or prevention of such a disease or disorder.

By “therapeutic regimen” is meant the pattern of treatment of an illness, e.g., the pattern of dosing used during the treatment of the disease or disorder.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where an FXR agonist, such as tropifexor, and the one or more additional therapeutic agents may be administered independently at the same time or separately within time intervals, especially where these time intervals allow that the combination partners show a cooperative, e.g. synergistic effect.

As herein defined, “combination” refers to either a fixed combination in one unit dosage form (e.g., capsule, tablet, or sachet), free (i.e. non-fixed) combination, or a kit of parts for the combined administration where an FXR agonist tropifexor of the present invention and licogliflozin or a pharmaceutically acceptable salt or solvate thereof, also referred to as or “co-agent”) may be administered independently at the same time or separately within time intervals, especially where these time intervals allow that the combination partners show a cooperative, e.g. synergistic effect.

The terms “co-administration” or “combined administration” or the like as utilized herein are meant to encompass administration of the additional therapeutic agent to a single subject in need thereof (e.g. a patient), and the additional therapeutic agent are intended to include treatment regimens in which the FXR agonist tropifexor and licogliflozin are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients, i.e. i) a non-bile acid derived FXR agonist tropifexor (in free form or e.g. as a pharmaceutically acceptable salt or an amino acid conjugate thereof) and ii) licogliflozin (as herein defined), are both administered to a patient simultaneously in the form of a single entity or dosage.

The term “free combination” means that the active ingredients as herein defined are both administered to a patient as separate entities either simultaneously, concurrently or sequentially with no specific time limits, and in any order, wherein such administration provides therapeutically effective levels of the two compounds in the body of the patient.

By “simultaneous administration”, it is meant that the FXR agonist tropifexor and licogliflozin (as herein defined), are administered on the same day. The two active ingredients can be administered at the same time (for fixed or free combinations) or one at a time (for free combinations).

According to the invention, “sequential administration”, may mean that during a period of two or more days of continuous co-administration only one of tropifexor and licogliflozin, is administered on any given day.

By “overlapping administration”, it is meant that during a period of two or more days of continuous co-administration, there is at least one day of simultaneous administration and at least one day when only one of tropifexor and licogliflozin, is administered.

By “interval administration”, it is meant a period of co-administration with at least one void day, i.e. with at least one day where neither tropifexor nor licogliflozin, is administered.

By “continuous administration”, it is meant a period of co-administration without any void day. The continuous administration may be simultaneous, sequential, or overlapping, as described above.

As used herein, the term “qd” means a once daily administration.

The term “dose” refers to a specified amount of a drug administered at one time. As used herein, the dose is the amount of the drug that elicits a therapeutic effect. The dose would, for example, be declared on a product package or in a product information leaflet. For example, the term “dose” when used in relation to tropifexor is the amount of tropifexor in free form. Since tropifexor can be present in the form of a salt or of an amino acid conjugate, the amount of the respective salt former (e.g. the respective acid) or of the amino acid, has to be added accordingly. When used in relation to licogliflozin, the term “dose” is the amount of licogliflozin in free form. Since licogliflozin can be present in the form of a salt or of a co-crystal, e.g. co-crystal with L-proline as herein defined, the amount of the respective salt former (e.g. the respective acid) or of L-proline, has to be added accordingly.

As used herein, the term “a,” “an,” “the” and similar terms used in the context of the present invention (especially in the context of the claims) are to be construed to cover both the singular and plural unless otherwise indicated herein or clearly contradicted by the context.

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Timing of the Administration

In one embodiment, the FXR agonist as herein defined in above listed embodiments, is administered in the evening. In another embodiment, the SGLT inhibitor, e.g. SGLT 1/2 inhibitor, as herein defined in above listed embodiments, is administered in the evening. In yet another embodiment, the FXR agonist and the SGLT inhibitor as herein defined in above listed embodiments, is administered in the evening.

The term “administration in the evening” is generally defined as administration any time from about 6 pm to about 12 pm, e.g. from about 8 pm to about 11 pm, preferably around 9 pm. Administration in the evening may be before the evening meal, with the evening meal or after the evening meal. In one embodiment, the term “administration in the evening” refers to administration shortly before or at bedtime. In some examples, the term “administration in the evening” refers to administration shortly before bedtime. In other examples, the term “administration in the evening” refers to administration at bedtime. Unless otherwise specified herein, the term “bedtime” has the normal meaning of a time when a person retires for the primary sleep period during a twenty-four hour period of time. The administration shortly before bedtime means that the FXR agonist or the SGLT inhibitor, e.g. SGLT 1/2 inhibitor as herein defined, is administered within about 30 minutes to about 2 hours prior to a person's normal rest or sleep (typically 4 to 10-hours) period.

### Diseases

As hereinabove defined, the fibrotic or cirrhotic disease or disorder can be a liver disease or disorder, e.g. as defined herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis. The liver diseases or disorders can also refer to liver transplantation.

As hereinabove defined, the intestinal disease can be idiopathic inflammatory bowel disease, e.g. Crohn's disease or ulcerative colitis.

In one embodiment of the invention, the pharmaceutical combinations (as herein defined) are for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a pharmaceutical combination of the invention, as herein defined in above listed embodiments for the improvement of liver fibrosis without worsening of steatohepatitis.

In another embodiment of the invention, there is provided a pharmaceutical combination as herein defined in above listed embodiments, for obtaining a complete resolution of steatohepatitis without worsening, e.g. improving liver fibrosis.

In another embodiment of the invention, there is provided a pharmaceutical combination as herein defined in above listed embodiments, for preventing or treating steatohepatitis and liver fibrosis.

In yet another embodiment of the invention, there is provided a pharmaceutical combination as herein defined in above listed embodiments, for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a pharmaceutical combination as herein defined in above listed embodiments, for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a pharmaceutical combination as herein defined, for treating or preventing, stage 3 fibrosis to stage 1 fibrosis, e.g. stage 3 and/or stage 2 and/or stage 1 fibrosis.

### Subjects

According to the invention, the subjects receiving the pharmaceutical combination of the invention can be affected or at risk of a fibrotic disease or disorder, e.g. a liver disease or disorder, e.g. as hereinabove defined.

In some embodiments of the invention, the subject is obese or overweight.

In other embodiments of the invention, the subject may be a diabetic subject, e.g. may have type 2 diabetes. The subject may have high blood pressure and/or high blood cholesterol level.

### Dosing Regimens

Depending on the compound used, the targeted disease or disorder and the stage of such disease or disorder, the dosing regimen, i.e. administered doses and/or frequency of each component of the pharmaceutical combination may vary.

The dosing frequency will depend on; inter alia, the phase of the treatment regimen.

The frequency of dosing of tropifexor and licogliflozin, e.g. as a fixed dose combination, may be once per day.

According to the invention, tropifexor and licogliflozin, both as herein defined, may not be administered following the same regimen, i.e. may not be administered at the same frequency and/or duration and/or dosage, e.g. at the same frequency and/or dosage. This can be the case e.g. for free combinations.

In one embodiment of the invention, the co-administration is carried out for at least one week, at least one month, at least 6 weeks, at least three months, at least 6 months, at least one year. For example, the pharmaceutical combination of the invention is administered lifelong to the patient. The frequency of administration, and/or the doses of the tropifexor and of licogliflozin, may vary during the whole period of administration.

In case of a sequential co-administration, tropifexor (as hereinabove defined) may be administered prior to licogliflozin (as hereinabove defined), or reciprocally. The time interval between administration of tropifexor and of licogliflozin may vary from a few minutes to a few days, e.g. a few minutes, e.g. a few hours, e.g. 1 day to 1 week.

In some aspects, tropifexor (as herein above defined) that is administered with licogliflozin (in free form or as a pharmaceutically acceptable salt, solvate, prodrug and/or ester thereof), is administered at a dose of about 120 μg, about 140 μg, or about 200 μg. Such doses may be for daily administration. Such doses are particularly adapted for oral administration of tropifexor.

According to the invention, tropifexor (as hereinabove defined), is administered at a dose of about 90 μg to about 250 μg, e.g. about 140 μg to about 200 μg, e.g. about 140 μg. Such doses may be for oral administration. Preferably, tropifexor (as hereinabove defined), is administered at a dose of about 90 μg, or about 140 μg.

In some aspects, tropifexor (as hereinabove defined), is administered at a dose of about 90 μg, about 100 μg, about 110 μg, about 120 μg, about 140 μg, or about 200 μg. Such doses are particularly adapted for oral administration of tropifexor.

In some embodiments, tropifexor, as herein defined, is administered at a dose of about 120 μg delivered orally, of about 140 μg delivered orally or of about 200 μg delivered orally.

In some embodiments, tropifexor as herein defined, is to be administered at a daily dose of about 90 μg; about 120 μg; about 140 μg; or about 200 μg.

Obeticholic acid is to be administered at a daily dose of about 5 mg, of about 10 mg, of about 15 mg, of about 20 mg, of about 25 mg, of about 30 mg, of about 40 mg or of about 50 mg. In some embodiments, obeticholic acid as herein defined, is to be administered at a daily dose of about 25 mg.

According to the invention, licogliflozin (as hereinabove defined) is administered at a dose of about 20 mg, e.g. about 30 mg, e.g. about 50 mg, e.g. about 60 mg, e.g. about 80 mg, e.g. about 90 mg, e.g. about 100 mg, e.g. about 120 mg, e.g. about 150 mg. Such doses may be for oral administration licogliflozin. Such doses may be for daily oral administration of licogliflozin.

In some aspects, licogliflozin (as herein above defined) is administered at a dose of about 30 mg. Such dose may be for daily oral administration licogliflozin.

### EXAMPLES

### Example 1

### A 2 Week Study in Cynomolgus Monkey Treated with an FXR Agonist

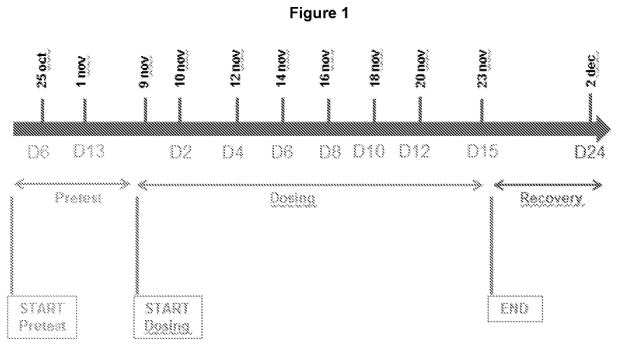

The rate of total bile acid production and the major subsets of the different bile acids have been measured in a 2 week study in Cynomolgus monkey treated with an FXR agonist (LJP305), as shown in FIG. 1 and described in Table 1.

TABLE 1
Study design.

| | No. of | Dose | Dose |
|---|---|---|---|
| | Animals | Level | Concentration |
| Group | Male | (mg/kg/day) | (mg/mL) |
| Phase I | | | |
| 1 LJP305-NX-11 (Low) | 4 | 0.1 | 0.02 |
| 2 LJP305-NX-11 (Mid) | 4 | 1 | 0.2 |
| 3 LJP305-NX-11 (High) | 4 | 3 | 0.6 |

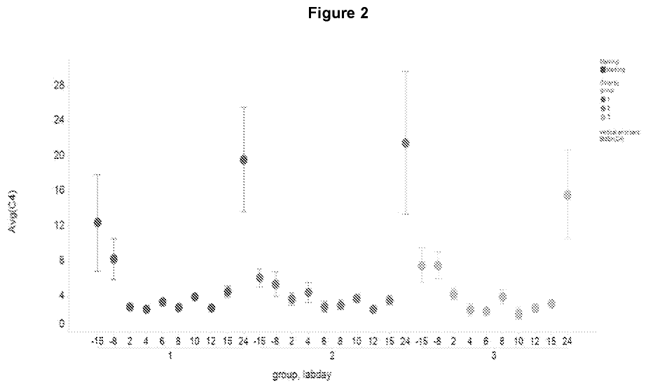

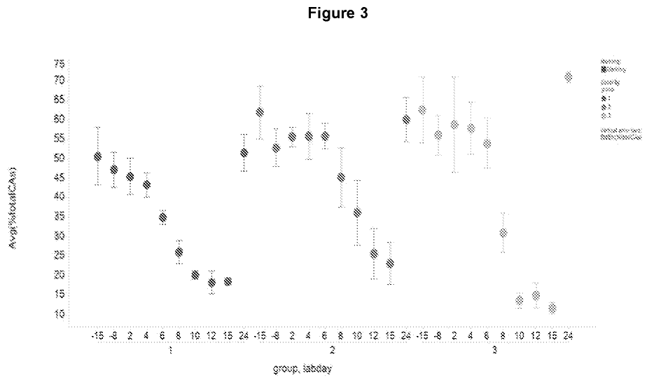

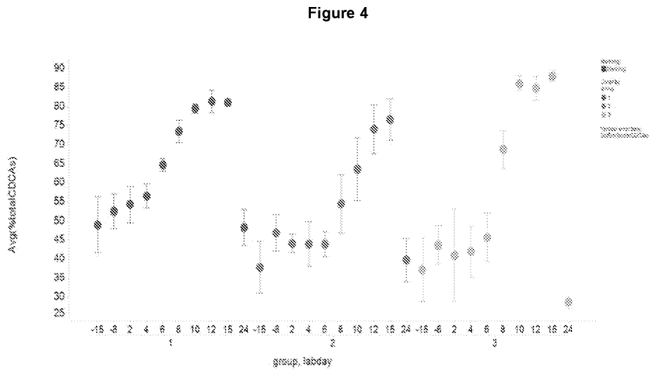

Although total bile acids were decreased (FIG. 2), the ratio of CA to CDCA bile acid was altered overtime with a severe decrease in CA (FIG. 3) but a concomitant increase in CDCA bile acids (FIG. 4).

The most effective method to avoid such an inhibition of Cyp7A1 and consequent activation of the alternate pathway would be to administer an FXR agonist when the enzymatic activity of Cyp7A1 is at the lowest in order to minimize the effect of an FXR-mediated inhibition of the Cyp1A1. As the activity of this enzyme is at the lowest in human during the night, administration of the FXR agonist in the evening (from about 6 pm to about 12 pm, e.g. from about 8 pm to about 11 pm, preferably around 9 pm) should coincide with the time the body naturally decreases the enzyme production/activity and consequently should minimize the impact of such inhibition hence reducing the chance of stimulating the alternate pathway with the resulting production of prurigenic bile acid (CDCA).

### Example 2

### In Vitro Human Hepatocytes Treated with FXR Agonists

FXR agonist treatments have been associated, in human, with lipid abnormalities, including increases in peripheral LDL. Increased cholesterol in hepatocytes is associated with a counter mechanism of decrease LDL receptor on the surface of the cells. Such a decrease in the LDL receptor on the surface of the hepatocytes will ultimately results in increases in circulating LDL; the phenotype observed in the clinics.

FIG. 5 shows that in vitro, using in vitro human hepatocytes, the FXR agonists, such as obeticholic acid (OCA) and cilofexor (GS-9674), reduce the LDL uptake by hepatocytes in a dose dependent manner. Those data indicates that blocking of the Cyp7A1 and the bile acid pathway leads to the peripheral increase in LDL. To mitigate the increase in peripheric LDL, we hypothesize that treating the subjects in the evening (from about 6 pm to about 12 pm, e.g. from about 8 pm to about 11 pm, preferably around 9 pm) would be reduce the impact of the drug on LDL. At such time of the day, the level of CYP7A1 are the lowest hence the FRX agonist would have little to no substrate to inhibit hence the inhibition of cholesterol excretion would be at its minimal. In addition, during night time, the hepatocytes rely less on cholesterol coming from the food intake (LDL and others) since the body is then fasting but more on the intrahepatic production of cholesterol via HMGCOa reductase; the activity of this enzyme is the highest during the night. Indeed, in human, whilst the Cyp7A1 activity peak at 1 and 9 pm, intracellular cholesterol levels in hepatocytes are the highest during the night (between midnight and 4 AM).

For high efficacy and/or good safety (e.g. a low risk of itch and/or lipid abnormalities), administration of FXR agonists in the evening is suggested.

### Example 3

### Clinical Study for Efficacy, Safety, and Tolerability in Subjects with NASH and Fibrosis (Stage 2 or 3) as Per NASH CRN Histological Score

Primary objective: To demonstrate the efficacy of the combination of tropifexor with licogliflozin, as assessed by histologic improvement after 48 weeks of treatment in subjects with NASH and stage 2 or 3 fibrosis.

### Secondary Objectives:

Improvement in fibrosis by at least one stage with no worsening of NASH after 48 weeks of treatment
Resolution of NASH with no worsening of fibrosis after 48 weeks of treatment
Improvement in fibrosis by at least one stage
Improvement in fibrosis by at least two stages with no worsening of NASH after 48 weeks of treatment
Reduction in body weight from baseline after 48 weeks of treatment
Change in liver fat content after 48 weeks of treatment
To determine the relationship of investigational treatment and markers of hepatic inflammation in NASH (ALT and AST)
To determine the relationship of investigational treatment and GGT, a marker of cholestasis

The study consists of 1) a screening period, 2) a treatment period starting from randomization on Day 1 and running to Week 48, and 3) a follow up period of 4 weeks after the last dose of study treatment. The screening period starts from the time of the signing of informed consent and continues for up to 8 weeks when all inclusion/exclusion criteria have been evaluated and all baseline assessments have been performed. The study duration from first dose of study medication is 52 weeks. The total duration of participation may be up to 60 weeks.

Subjects eligible for inclusion in this study must meet all of the following criteria:
Written informed consent must be obtained before any assessment is performed.
Male and female subjects 18 years or older (at the time of the screening visit)
Presence of NASH as demonstrated by the following during the screening period: NASH with fibrosis stage 2 or 3 confirmed by central reader's evaluation using NAFLD Activity Score (NAS) and NASH CRN criteria, of liver biopsy obtained no more than 6 months before randomization.
Able to communicate well with the investigator, to understand and comply with the requirements of the study

The planned duration of treatment is 48 weeks. Subjects may be discontinued from treatment earlier due to unacceptable tolerability, disease progression and/or at the discretion of the investigator or the subject.

In a Phase 2 dose range finding study in obese patients, licogliflozin was associated with a dose-dependent increase in incidence of diarrhea (18.4%, 15.8%, 55.3%, 68.8%, following 2.5, 10, 50, and 150 mg QD for 24 weeks vs. 19.2% on placebo; CLIK066B2201). A dose of 30 mg QD is expected to achieve approximately 70% of maximum observed efficacy (using weight loss as a downstream marker for efficacy; CLIK066B2201). Licogliflozin is currently being tested as 30 mg and 150 mg QD monotherapy in NASH (CLIK066X2204). An interim analysis on the 150 mg QD treatment group showed promising reduction of ALT and liver fat, among other efficacy endpoints, but significant incidence of gastrointestinal events (mainly diarrhea). To minimize the risk of GI adverse effects of the SGLT1 inhibition in the gut such as diarrhea, licogliflozin will be administered in the evening.

The doses for this study were selected based on the expectation of achieving increased efficacy with the combination therapy, compared to individual monotherapies, while maintaining tolerability and safety of the patients.

Subjects (n=70) are assigned at baseline visit to Combination therapy Arm: tropifexor 140 μg+licogliflozin 30 mg, once daily. Subjects should take the medication in the evening following a meal and at about the same time each day, except at baseline and week 4 where the dose will be taken in the morning at the clinic instead of evening dose.

The efficacy assessments should be completed in the following recommended order:
MRI.
Liver function test: ALT, AST, GGT, total alkaline phosphatase (and isoenzymes if total alkaline phosphatase is >ULN, and 5′nucleotidase if either GGT or total alkaline phosphatase is >ULN during study participation), total bilirubin, and albumin will be assessed.
Protein measurements using SOMAscan®.
Markers of liver fibrosis: originally called Fibrotest®/Fibrosure®. The following will be assessed: a2-macroglobulin, apolipoprotein A1, total bilirubin, haptoglobin, GGT, and ALT.
NAFLD fibrosis score: The following formula will be utilized for the calculation of the NAFLD fibrosis score: −1.675+0.037×age (years)+0.094×BMI (kg/m2)+1.13×IFG (increased fasted glucose)/diabetes (yes=1, no=0)+0.99×AST/ALT ratio −0.013×platelet (×109/l)−0.66×albumin (Op.
Fasting insulin and glucose: Blood samples will be collected for fasting insulin and glucose assessment.
Liver biopsy: Subjects must have histologic evidence of NASH and liver fibrosis stage 2 or 3 (NASH clinical research network (CRN) staging criteria) demonstrated on liver biopsy within 6 months prior to randomization.

In addition, a Transient Elastography (FibroScan®) can be done at screening/baseline and at the Week 12, 24 and, 48. Standard safety parameters and measures are collected including adverse events and serious adverse events according to definitions and process detailed in the protocol.

### Example 4

### Safety, Tolerability and Efficacy of Licogliflozin, an SGLT1/2 Inhibitor in Patients with Non-Alcoholic Fatty Liver Disease: Interim Analysis of a Placebo-Controlled, Randomized Phase 2a Study

A randomized, double blinded, placebo-controlled Phase 2a study was conducted to evaluate the safety, tolerability and efficacy of licogliflozin in patients with either histologically confirmed NASH or with a biochemical phenotype suggestive of NASH.

METHOD: Patients with histologically confirmed NASH (F1-F3) or phenotypic NASH (BMI≥27 kg/m² in non-Asians or 23 kg/m² in Asians, ALT≥50 (males) or 35 (females) and type 2 diabetes (T2DM)) received daily oral licogliflozin at 150 mg, 30 mg or placebo in a 2:2:1 ratio for 12 weeks (NCT03205150). The primary endpoint is the effect on ALT level after 12 weeks of treatment. Secondary endpoints include improvement in body weight, liver fat content and AST, amongst others. The study size is 110 of which 77 have completed (placebo (n=18); licogliflozin 30 mg (n=25) and licogliflozin 150 mg (n=34)) and are included in the interim analysis.

RESULTS: After 12 weeks of treatment, there was a 27% (17.2 U/L, p=0.036) and 19% (11.1 U/L, p=NS) placebo adjusted reduction from baseline levels of ALT at 150 mg and 30 mg, respectively. There was a reduction in AST of 30% (p=0.004) and 23% (p=0.043) as well as a 32% (p=0.001) and 26% (p=0.014) in GGT at 150 mg and 30 mg doses, respectively. Placebo adjusted reductions in body weight at both doses (˜4%, p=0.0001) and HbA1c (absolute change: 150 mg, 0.96% (p=0.0001); 30 mg, 0.81% (p=0.001)) were seen. Relative reduction in liver fat content was 22% (p=0.01) and 10% (p=NS) at 150 mg and 30 mg, respectively, and the proportion of patients with at least a 30% relative reduction was 66.7% (150 mg), 39.5% (30 mg) and 25% (placebo). Absolute reduction in liver fat was 4.45% (p=0.01) at 150 mg and 2.71% (p=NS) at 30 mg with 63.3% (150 mg), 43.5% (30 mg) and 18.8% (placebo) of patients achieving at least 5% absolute reduction. Diarrhea, the most common adverse event (AE), was reported by similar number of patients in the placebo and 30 mg group (38.9% vs. 40%) but was higher at the 150 mg dose (76.5%). Most diarrhea events (97.4%) were mild.

The study showed that Licogliflozin is safe and tolerable and improves multiple biochemical endpoints associated with NASH after 12 weeks of treatment. The study achieved its primary end-point of statistically significant reduction in ALT of at least 25% compared to placebo as showed above (mean relative decrease in ALT of 27% and 19% versus placebo at 150 mg and 30 mg, respectively and statistically significant reductions in AST and GGT versus placebo at both doses).

### Example 5

### Role of Tropifexor in the Reductions of Hepatic Fat and Serum Alanine Aminotransferase in Patients with Fibrotic NASH After 12 Weeks of Therapy (FLIGHT-FXR Part C interim Results)

Parts A and B of study CLJN452A2202 in NASH patients have investigated tropifexor at doses ranging from 10 to 90 μg daily for 12 weeks. Tropifexor exhibited a clear dose response for target engagement (FGF19) and biologic activity (GGT). ALT and hepatic fat fraction were reduced across all tropifexor doses (10, 30, 60 and 90 μg) compared to placebo. The study showed that Tropifexor was generally well tolerated up to 90 μg daily without safety signals. Results from the first two parts (A and B, study CLJN452A2202) demonstrated anti-inflammatory and anti-steatotic efficacy of 60 and 90 μg of tropifexor based on biomarkers, and favorable safety at Week 12.

FLIGHT-FXR (NCT02855164) is a phase 2 randomized, double blind, placebo-controlled, 3-part, adaptive-design study to assess the safety, tolerability, and efficacy of several doses of tropifexor (LJN452) in patients with non-alcoholic steatohepatitis (NASH).
METHODS: In Part C, the effects of higher doses of tropifexor on biomarkers and histology will be evaluated over 48 weeks in patients with biopsy-proven NASH and fibrosis stages 2-3. In all, 152 patients (64% females) were randomized to receive placebo (N=51), tropifexor 140 μg (N=50) or tropifexor 200 μg (N=51) once daily. Pre-specified endpoints assessed at week 12 included overall safety and changes in alanine aminotransferase (ALT), hepatic fat fraction (HFF), gamma glutamyl transferase (GGT), and body weight.
RESULTS: Pre-specified endpoints were met for tropifexor at a dose of 200 μg. Efficacy results are presented in Table 2.

TABLE 2
Least squares means of absolute changes in ALT, GGT, and body
weight, and relative change in HFF from baseline to
Week 12 estimated in repeated measures or analysis of
covariance models (full analysis set)

| | | Tropifexor | Tropifexor |
|---|---|---|---|
| Biomarkers | Placebo (N = 51) | 140 μg (N = 50) | 200 μg (N = 51) |
| ALT (U/L) | −8.9 (4.19) | −20.1 (4.57) | −23.6 (4.48) |
| | n = 49 | n = 41; | n = 39; |
| | | P = 0.058 | P = 0.013 |
| Relative change | −10.26 (4.21) | −16.99 (4.64) | −31.37 (4.30) |
| in HFF* (%) | n = 51 | n = 49; | n = 51; |
| | | P = 0.209 | P < 0.001 |
| GGT (U/L) | −2.5 (3.55) | −39.2 (3.70) | −40.9 (3.62) |
| | n = 49 | n = 44; | n = 46; |
| | | P < 0.001 | P < 0.001 |
| Body weight (kg) | −1.14 (0.36) | −2.46 (0.38) | −3.20 (0.37) |
| | n = 50 | n = 46; | n = 46; |
| | | P = 0.010 | P < 0.001 |

*Measured as magnetic resonance imaging-proton density fat fraction (MRI-PDFF).
Data are presented as LS mean change (SE) with 2-sided P values reported for statistical significance
ALT, alanine aminotransferase;
GGT, gamma glutamyl transferase;
HFF, hepatic fat fraction;
LS, least square;
SE, standard error;

Relative HFF reduction (without imputation for missing values) by ≥30% was achieved in 20%, 32%, and 64% of patients in the placebo, Tropifexor 140 μg, and Tropifexor 200 μg groups, respectively. The frequency of serious adverse events was low and comparable across groups. Among patients with pruritus, >60% in both Tropifexor groups and all in the placebo group experienced events with mild (Grade 1) severity. Treatment discontinuation rates due to pruritus were low (Tropifexor 140 μg: n=1 [2%]; Tropifexor 200 μg: n=3 [6%]; placebo: 0%). A dose-related increase in low density lipoprotein-cholesterol (LDL-C) was seen. None of the lipid changes led to treatment discontinuation or dose reduction.

In this pre-specified interim analysis of Part C, higher doses of Tropifexor resulted in robust and dose-dependent decreases in ALT, HFF, and body weight with good safety and tolerability after 12 weeks of treatment. Similar to other FXR agonists, these higher doses were associated with mild pruritus and minor dose-related increase in LDL-C.

### Example 6

### Dose-Dependent Reduction in Body Weight with Licogliflozin Treatment in Patients with Obesity Disease

This study was a randomized, double-blind, placebo controlled, dose finding study to evaluate the effect of licogliflozin (2.5, 10, 25 and 50 mg qd) in 126 Japanese patients with obesity disease. The primary objective was to examine the dose-response relationship of licogliflozin treatment in body weight reduction relative to placebo at 12 weeks. The secondary objectives included assessment of responder rates, change in parameters related to complications, visceral and subcutaneous fat area, and safety through 12 weeks of treatment.

RESULTS: The placebo-subtracted percentage change in body weight from baseline at Week 12 was −1.99, −3.00, −3.54, and −3.91% in licogliflozin 2.5, 10, 25 and 50 mg qd dose groups, respectively. In total, ≥50% of patients achieved reduction of ≥3% in body weight in licogliflozin 10, 25 and 50 mg qd dose groups versus placebo (7.1%; p≤0.002 for all). Treatment with licogliflozin was safe with no ketoacidosis and no new safety signals. Dual inhibition of SGLT1/2 with licogliflozin treatment induced a dose-dependent reduction in body weight in Japanese patients with obesity disease. Administration of licogliflozin (2.5, 10, 25 and 50 mg qd) over 12 weeks was safe and well tolerated in this study.

### Example 7

### Safety and Drug-Drug Interaction (DDI) of Tropifexor in Combination with Licogliflozin in Healthy but Overweight to Obese Subjects

Method: This was a single site, 3-period, open-label, multiple dose, fixed-sequence, cross-over study to evaluate the DDI between tropifexor and licogliflozin in healthy but overweight to obese subjects under fed conditions (standard meal). Sixteen subjects with a BMI of 25 to 35 kg/m2 were enrolled. The study consisted of a) tropifexor 140 μg treatment for 4 days (Period 1) followed by a 7-day washout period, b) licogliflozin 50 mg treatment for 7 days (Period 2) directly followed by c) combined treatment with tropifexor 140 μg and licogliflozin 50 mg for 4 days (Period 3). Standard safety assessments were performed, with adverse event monitoring. Steady state PK parameters from Period 1 or 2 (single agent PK) were compared with that from Period 3 (combination PK) to assess the effect of potential drug interaction.

Results: Combination of tropifexor and licogliflozin was well tolerated and safe in the overweight to obese subjects. Seven mild or moderate adverse events were reported with the mono-therapy treatments, while no AEs were observed during the combination treatment. No meaningful DDI was observed in the study. In the presence of licogliflozin, mean peak plasma concentrations (Cmax,ss) of tropifexor decreased by 15% and total exposure (AUCtau,ss) decreased by 12%. In the presence of tropifexor, mean peak plasma concentrations of licogliflozin increased by 8% and total exposure increased by 12%, all within the 0.8-1.25 bioequivalence range. In conclusion, the study data support the doses selected for the study of Example 3.

### Example 8

### A Randomized, Investigator and Subject Blinded, Multi-Center, Parallel Arm Study to Determine the Safety and Tolerability of Tropifexor Administered in the Morning or in the Evening to Subjects with NASH

The objective of this study is to determine the effect of tropifexor dosed AM or PM on fasting circulating LDL-C levels, HDL-C after 2 weeks/4 weeks of treatment.

The study consists of a screening period up to 14 days, baseline period up to 21 days, treatment period of 4 weeks followed by a study completion evaluation approximately 30 days after the end of the treatment period. The study population is comprised of male and female adult overweight or obese subjects with EITHER histologic evidence of NASH on liver biopsy within 2 years prior to screening OR phenotypic diagnosis of NASH based on elevated ALT and BMI, diagnosis of Type 2 diabetes (T2D) or currently taking anti-diabetic medications and liver fat content 5% by MRI-PDFF. This study investigates if dosing tropifexor in the evening could have advantages over dosing in the morning both in terms of effect on lipids and on pruritus.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A pharmaceutical combination for simultaneous, sequential or separate administration, comprising (i) an FXR agonist selected from tropifexor, obeticholic acid, nidufexor, cilofexor, TERN-101, EDP-305, PXL007, AGN242266 and MET409; and (ii) an SGLT inhibitor, e.g. SGLT 1/2 inhibitor.

2. The pharmaceutical combination according to claim 1, wherein the FXR agonist is tropifexor.

3. The pharmaceutical combination according to claim 2, comprising (i) an amount of 90 μg to about 250 μg, or about 140 μg to about 200 μg of tropifexor.

4. The pharmaceutical combination according to claim 3, comprising an amount of about 140 μg of tropifexor.

5. The pharmaceutical combination according to claim 1, wherein the SGLT inhibitor is selected from licogliflozin, dapagliflozin, canagliflozin, empagliflozin, ipragliflozin, ertugliflozin, mizagliflozin, sotagliflozin.

6. The pharmaceutical combination according to claim 5, wherein the SGLT inhibitor is licogliflozin.

7. The pharmaceutical combination according to claim 6, comprising ii) an amount of about 30 mg or 50 mg of licogliflozin.

8. The pharmaceutical combination according to claim 1, comprising: (i) about 90 pg of tropifexor; and (ii) about 30 mg of licogliflozin.

9. The pharmaceutical combination according to claim 1, comprising: (i) about 140 μg of tropifexor; and (ii) about 30 mg of licogliflozin.

10. The pharmaceutical combination according to claim 6, wherein said licogliflozin is an L-proline salt of licogliflozin.

11. The pharmaceutical combination according to claim 6, wherein said licogliflozin is an L-proline co-crystal of licogliflozin.

12. The pharmaceutical combination according to claim 11, wherein the L-proline co-crystal of licogliflozin has a 1:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol.

13. The pharmaceutical combination according to claim 11, wherein the L-proline co-crystal of licogliflozin has a 2:1 molar ratio of L-proline to (2S,3R,4R,5S,6R)-2-[3-(2,3-dihydro-benzo[1,4]dioxin-6-ylmethyl)-4-ethyl-phenyl]-6-hydroxymethyl-tetrahydropyran-3,4,5-triol.

14. (canceled)

15. A method of preventing, delaying or treating a liver disease or disorder, in a subject in need thereof, comprising administering a therapeutically effective amount of the pharmaceutical combination according to claim 1.

16. The method according to claim 15, wherein the liver disease or disorder is a fibrotic or cirrhotic liver disease or disorder, selected from the group consisting of non-alcoholic fatty liver disease (NAFLD), non-alcoholic steatohepatitis (NASH), liver cirrhosis, alcohol-induced cirrhosis, cystic fibrosis-associated liver disease (CFLD), liver fibrosis, and progressive fibrosis of the liver caused by any of the diseases above or by infectious hepatitis.

17. The method according to claim 15, wherein the liver disease or disorder is non-alcoholic fatty liver disease (NAFLD), non-alcoholic steatohepatitis (NASH), primary biliary cirrhosis (PBC), liver fibrosis, or liver cirrhosis.

18. The method according to claim 16, wherein the liver disease or disorder is non-alcoholic fatty liver disease, (NAFLD) or non-alcoholic steatohepatitis (NASH).

19. The method according to claim 15, wherein said FXR agonist is administered in the evening; or said SGLT inhibitor is administered in the evening; or both said FXR agonist and said SGLT inhibitor are administered in the evening.

20. A method for treating a condition mediated by Farnesoid X receptor (FXR) in a subject in need thereof, comprising administering to said subject a pharmaceutical combination comprising:
(i) an FXR agonist, wherein the FXR agonist is administered once daily at a therapeutically effective dose, and wherein the FXR agonist is administered in the evening, and
(ii) an SGLT inhibitor, e.g. SGLT 1/2 inhibitor;
wherein said condition mediated by FXR is non-alcoholic fatty acid liver disease (NAFLD) or non-alcoholic steatohepatitis (NASH).
